(19) United States
(12) Patent Application Publication
(10) Pub. No.: US 2011/0060385 A1
(43) Pub. Date: Mar. 10, 2011

(54) DETERMINING STIMULATION LEVEL PARAMETERS IN IMPLANT FITTING

(76) Inventor: Sean K. Lineaweaver, Parker, CO (US)

(21) Appl. No.: 12/879,727

(22) Filed: Sep. 10, 2010

Related U.S. Application Data
(63) Continuation-in-part of application No. 12/557,242, filed on Sep. 10, 2009.

(51) Int. Cl. A61F 11/04; A61N 1/36

(52) U.S. Cl. 607/57

(57) ABSTRACT

A fitting system that may be used by a recipient to determine stimulation level parameters, such as threshold and/or maximum comfort levels, for a stimulating medical device is provided. These parameters may be for MAPs that may be used by a genetic algorithm in fitting a stimulating medical device. In obtaining these parameters, an internal component implanted in a recipient may apply stimulation to the recipient. In response, the recipient, using a user interface, may their information regarding their perception of the applied stimulation. This response may then be used to determine a stimulation level parameter that is then transmitted to the stimulation medical device for use in applying stimulation.

### RELATED APPLICATIONS

This application is a continuation-in-part of and claims priority to U.S. patent application Ser. No. 12/557,242, entitled “Determining Stimulation Level Parameters in Implant Fitting,” filed Sep. 10, 2009, the entire contents and disclosures which is hereby incorporated by reference.

### BACKGROUND

1. Field of the Invention

The present invention relates generally to stimulating medical devices, and more particularly, to fitting a stimulating medical device.

2. Related Art

Hearing loss, which may be due to many different causes, is generally of two types, conductive and sensorineural. In some cases, a person may have hearing loss of both types. Conductive hearing loss occurs when the normal mechanical pathways for sound to reach the cochlea, and thus the sensory hair cells therein, are impeded, for example, by damage to the ossicles. Conductive hearing loss is often addressed with conventional hearing aids which amplify sound so that acoustic information can reach the cochlea.

In many people who are profoundly deaf, however, the reason for their deafness is sensorineural hearing loss. Sensorineural hearing loss occurs when there is damage to the inner ear or to the nerve pathways from the inner ear to the brain. Those suffering from some forms of sensorineural hearing loss are unable to derive suitable benefit from conventional hearing aids. As a result, hearing prostheses that deliver electrical stimulation to nerve cells of the recipient's auditory system have been developed to provide the sensations of hearing to persons whom do not derive adequate benefit from conventional hearing aids. Such stimulating hearing prostheses include, for example, auditory brain stimulators and cochlear prostheses (commonly referred to as cochlear prosthetic devices, cochlear implants, cochlear devices, and the like; simply “cochlear implants” herein.) As used herein, the recipient's auditory system includes all sensory system components used to perceive a sound signal, such as hearing sensation receptors, neural pathways, including the auditory nerve and spiral ganglia, and regions of the brain that sense sound.

Sensorineural hearing loss is commonly due to the absence or destruction of the cochlear hair cells which transduce acoustic signals into nerve impulses. Cochlear implants help treat such sensorial hearing loss. Cochlear implants use direct electrical stimulation of auditory nerve cells to bypass absent or defective hair cells that normally transduce acoustic vibrations into neural activity. Such devices generally use an electrode array implanted into the scala tympani of the cochlea so that the electrodes may differentially activate auditory neurons that normally encode differential pitches of sound.

Auditory brain stimulators are used to treat a smaller number of recipients with bilateral degeneration of the auditory nerve. For such recipients, the auditory brain stimulator provides stimulation of the cochlear nucleus in the brainstem.

### SUMMARY

In one aspect of the present invention a method for fitting a stimulating medical device to a recipient is provided. This method comprises: transmitting a signal to cause the stimulating medical device to apply stimulation to the recipient; displaying a graphical user interface to the recipient; receiving a response to the applied stimulation from the recipient via the graphical user interface; determining stimulation level parameter using the recipient's response; and transmitting the stimulation level parameter to the stimulating medical device for use in applying stimulation to the recipient.

In another aspect of the present invention a system for fitting a stimulating medical device to a recipient is provided. This system comprises: a fitting system controller configured to transmit a signal to cause the stimulating medical device to apply stimulation to the recipient; a display configured to display a graphical user interface to the recipient; and an input device configured to receive a response from the recipient, using the graphical user interface, regarding stimulation applied by the stimulating medical device; wherein the fitting system controller is further configured to determine a stimulation level parameter using the received response, and transmit the determined stimulation level parameter to the stimulation medical device for use in applying stimulation.

In yet another aspect of the present invention a system for fitting a stimulating medical device to a recipient is provided. This system comprises: means for transmitting a signal to cause the stimulating medical device to apply stimulation to the recipient; means for displaying a graphical user interface to the recipient; means for receiving a response to the applied stimulation from the recipient via the graphical user interface; means for determining a stimulation level parameter using the recipient's response; and means for transmitting the stimulation level parameter to the stimulating medical device for use in applying stimulation to the recipient.

In yet another aspect of the present invention a computer readable medium comprising a computer program for controlling a processor to execute a method for fitting a stimulating medical device to a recipient is provided. This method comprises: transmitting a signal to cause the stimulating medical device to apply stimulation to the recipient; displaying a graphical user interface to the recipient; receiving a response to the applied stimulation from the recipient via the graphical user interface; determining a stimulation level parameter using the recipient's response; and transmitting the stimulation level parameter to the stimulating medical device for use in applying stimulation to the recipient.

### BRIEF DESCRIPTION OF THE DRAWINGS

Embodiments of the present invention are described below with reference to the attached drawings, in which:

FIG. 1 is a perspective view of a cochlear implant in which embodiments of the present invention may be implemented;

FIG. 2 is a schematic diagram illustrating one exemplary arrangement in which a recipient operated fitting system may be used to determine parameters for a stimulating medical device, in accordance with an embodiment;

FIG. 3 is a high-level flow chart illustrating operations that may be performed for measuring parameters for a stimulating medical device, in accordance with an embodiment;

FIG. 4 illustrates an exemplary GUI that may be provided to a recipient for obtaining the recipients perception of applied stimulation, in accordance with an embodiment;

FIG. 5A illustrates an exemplary GUI that may be provided to a recipient for measuring comfort levels, in accordance with an embodiment;

FIG. 5B illustrates another exemplary GUI that may be provided to a recipient for measuring comfort levels, in accordance with an embodiment;

FIG. 6 provides an exemplary GUI that may be used by a recipient to individually adjust electrode current levels, in accordance with an embodiment;

FIG. 7 illustrates an exemplary GUI that may be used by a recipient for balancing an electrode, in accordance with an embodiment; and

FIGS. 8A-8B illustrates an exemplary clinical graphical user interface that may used to add a MAP for level (e.g., T and C) measurement, in accordance with an embodiment.

### DETAILED DESCRIPTION

Aspects of the present invention are generally directed to a fitting system that may be used by a recipient to determine stimulation level parameters for a stimulating medical device. As used herein, “a stimulation level parameter” refers to any parameter regarding a stimulation level, such as, for example, threshold levels and/or maximum comfort levels for a stimulating medical device. For example, in an embodiment, the fitting system may be used by a recipient to determine the thresholds and maximum comfort levels for the possible MAPs that may be used by a genetic algorithm in fitting the stimulating medical device.

The automated fitting system may be provided with an instruction set specifying the parameter(s) to be measured (e.g., threshold or maximum comfort levels) as well as the characteristics of the stimulation to be applied in obtaining these measurements. As will be discussed in more detail below this instruction set may enable a recipient to measure their own stimulation level parameters (e.g., threshold and comfort levels). These measurements may be obtained, for example using psychophysical measurements in which stimulation is applied to the recipient in accordance with this instruction set. The fitting system may provide a graphical user interface (GUI) to the recipient that the recipient may use to provide an indication regarding their perception of the applied stimulation. For example, in an embodiment testing for threshold levels, the fitting system may apply multiple stimulation signals (perceived as beeps, if audible) of different current levels to the recipient. The fitting system may present a GUI to the recipient that includes a plurality of icons, each indicative of a particular number of beeps potentially heard by the recipient. The recipient may then select the icon representing the number of beeps heard. The fitting system may then implement an iterative procedure using this provided information to determine the threshold level for the stimulation channel being tested. In addition to measuring threshold levels, the fitting system may also be used in a similar manner for measuring maximum comfort levels for the stimulation channels, as well as other parameters.

Embodiments of the present invention are described herein primarily in connection with one type of hearing prosthesis, namely a cochlear prostheses (commonly referred to as a cochlear prosthetic devices, cochlear implant, cochlear devices, and the like; simply “cochlea implant” herein.) Cochlear implants generally refer to hearing prostheses that deliver electrical stimulation to the cochlea of a recipient. As used herein, cochlear implants also include hearing prostheses that deliver electrical stimulation in combination with other types of stimulation, such as acoustic or mechanical stimulation. It would be appreciated that embodiments of the present invention may be implemented in any cochlear implant or other hearing prosthesis now known or later developed, including auditory brain stimulators, or implantable hearing prostheses that acoustically or mechanically stimulate components of the recipient's middle or inner ear.

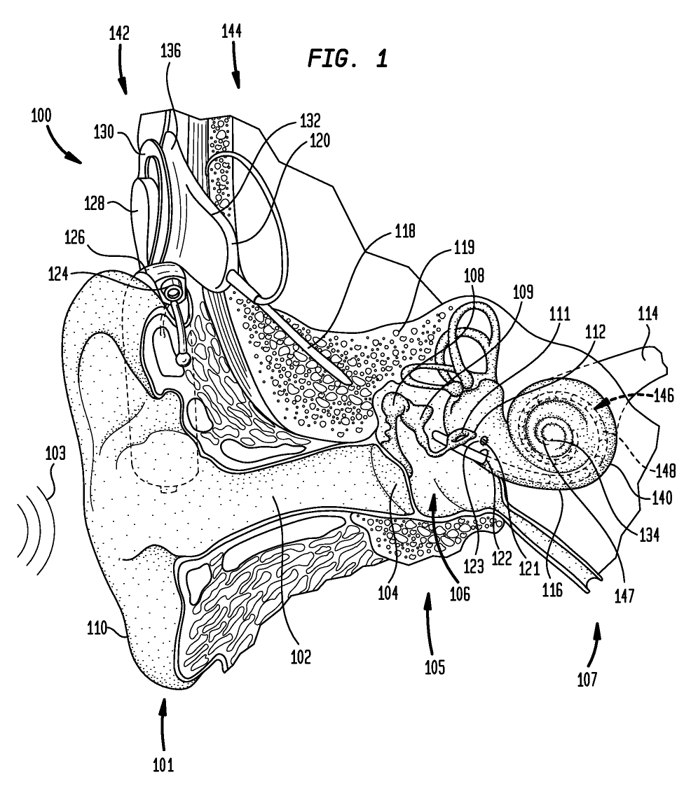

FIG. 1 is perspective view of a conventional cochlear implant, referred to as cochlear implant 100 implanted in a recipient having an outer ear 101, a middle ear 105 and an inner ear 107. Components of outer ear 101, middle ear 105 and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear cannel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

Cochlear implant 100 comprises an external component 142 which is directly or indirectly attached to the body of the recipient, and an internal component 144 which is temporarily or permanently implanted in the recipient. External component 142 typically comprises one or more sound input elements, such as microphone 124 for detecting sound, a sound processing unit 126, a power source (not shown), and an external transmitter unit 128. External transmitter unit 128 comprises an external coil 130 and, preferably, a magnet (not shown) secured directly or indirectly to external coil 130. Sound processing unit 126 processes the output of microphone 124 that is positioned, in the depicted embodiment, by auricle 110 of the recipient. Sound processing unit 126 generates encoded signals, sometimes referred to herein as encoded data signals, which are provided to external transmitter unit 128 via a cable (not shown).

Internal component 144 comprises an internal receiver unit 132, a stimulator unit 120, and an elongate electrode assembly 118. Internal receiver unit 132 comprises an internal coil 136, and preferably, a magnet (also not shown) fixed relative to the internal coil. Internal receiver unit 132 and stimulator unit 120 are hermetically sealed within a biocompatible housing, sometimes collectively referred to as a stimulator/receiver unit. The internal coil receives power and stimulation data from external coil 130, as noted above. Elongate electrode assembly 118 has a proximal end connected to stimulator unit 120, and a distal end implanted in cochlea 140. Electrode assembly 118 extends from stimulator unit 120 to cochlea 140 through mastoid bone 119, and is implanted into cochlea 104. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, sometimes referred to as electrode array 146 herein, disposed along a length thereof. Although electrode array 146 may be disposed on electrode assembly 118, in most practical applications, electrode array 146 is integrated into electrode assembly 118. As such, electrode array 146 is referred to herein as being disposed in electrode assembly 118. Stimulator unit 120 generates stimulation signals which are applied by electrodes 148 to cochlea 140, thereby stimulating auditory nerve 114.

In cochlear implant 100, external coil 130 transmits electrical signals (i.e., power and stimulation data) to internal coil 136 via a radio frequency (RF) link. Internal coil 136 is typically a wire antenna coil comprised of multiple turns of electrically insulated single-strand or multi-strand platinum or gold wire. The electrical insulation of internal coil 136 is provided by a flexible silicone molding (not shown). In use, implantable receiver unit 132 may be positioned in a recess of the temporal bone adjacent auricle 110 of the recipient.

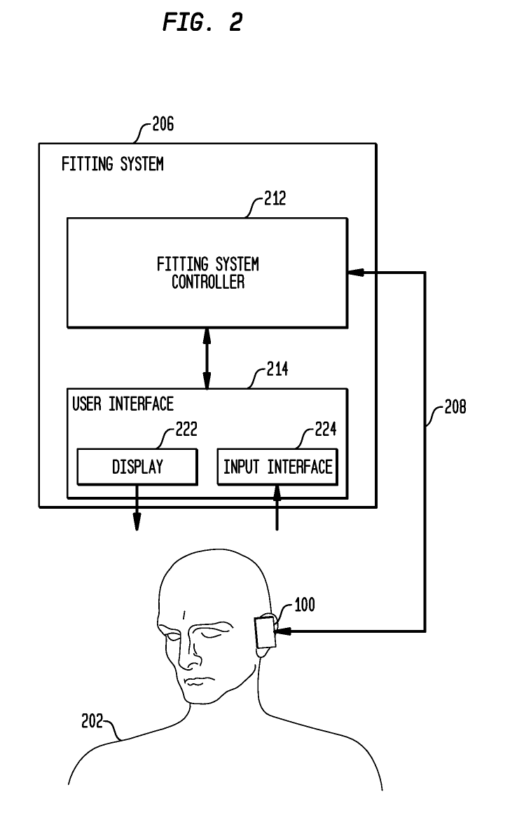

FIG. 2 is a schematic diagram illustrating one exemplary arrangement 200 in which a recipient 202 operated fitting system 206 may be used to determine stimulation level parameters (e.g., threshold and comfort levels) for a stimulating medical device 100, in accordance with an embodiment. In the embodiment illustrated in FIG. 2, sound processing unit 126 of cochlear implant 100 may be connected directly to fitting system 206 to establish a data communication link 208 between the sound processing unit 126 and fitting system 206. Fitting system 206 is thereafter bi-directionally coupled by means of data communication link 208 with sound processing unit 126. It should be appreciated that although sound processing unit 126 and fitting system 206 are connected via a cable in FIG. 2, any communications link now or later developed may be utilized to communicably couple the implant and fitting system.

Fitting system 206 may comprise a fitting system controller 212 as well as a user interface 214. Controller 212 may be any type of device capable of executing instructions such as, for example, a general or special purpose computer, digital electronic circuitry, integrated circuitry, specially designed ASICs (application specific integrated circuits), firmware, software, and/or combinations thereof. User interface 214 may comprise a display 222 and an input interface 224. Display 222 may be, for example, any type of display device, such as, for example, those commonly used with computer systems. Input interface 224 may be any type of interface capable of receiving information from a recipient, such as, for example, a computer keyboard, mouse, voice-responsive software, touch-screen (e.g., integrated with display 222), retinal control, joystick, and any other data entry or data presentation formats now or later developed.

Today, most cochlear implants require at least two stimulation level parameters to be set for each stimulating electrode 142. These values are referred to as the Threshold level (commonly referred to as the “THR” or “T-level;” “threshold level” herein) and the Maximum Comfortable Loudness level (commonly referred to as the Most Comfortable Loudness level, “MCL,” “M-level,” or “C;” simply “comfort level” herein). Threshold levels are comparable to acoustic threshold levels; comfort levels indicate the level at which a sound is loud but comfortable. It should be appreciated that although the terminology and abbreviations are device-specific, the general purpose of threshold and comfort levels is common across all cochlear implants: to determine a recipient's electrical dynamic range.

Because of the currently common usage of threshold and comfort levels, exemplary embodiments of the present invention are described herein in the context of determining such values for cochlear implant 100. As one of ordinary skill in the art would appreciate, however, the present invention may be used to measure stimulation level parameters for any prosthetic hearing implant now or later developed.

In contrast to conventional approaches in which an audiologist with specialized training in the field of cochlear implants obtains the stimulation level parameters (e.g., threshold and comfort levels), embodiments of the present invention enable a recipient without specialized knowledge to perform these measurements. As will be described in detail below, this is achieved by providing the recipient with a user interface that may receive information from the recipient regarding their perception of stimulation applied by the cochlear implant.

In addition to measuring stimulation level parameters, fitting system 206 may also be configured to interface with an application that searches for an optimum set of parameters for a cochlear implant 100 using a genetic algorithm. This set of parameters and their respective values is collectively and generally referred to herein as a “parameter map,” a “cochlear map” or “MAP.” A “MAP” is also sometimes referred to as a “program.” A further description of an exemplary genetic algorithm search that may be implemented by fitting system 206 is provided in U.S. patent application Ser. No. 12/557,208 entitled “Using a Genetic Algorithm to Fit A Cochlear Implant System to a Patient,” (Attorney Docket No.: 22409-00699) filed concurrent with the present application, which is incorporated herein in its entirety.

As noted, in embodiments, fitting system 206 may be used for determining the stimulation level parameters (e.g. threshold and comfort levels) as to perform a genetic algorithm search to determine the MAP to be implemented by the patient's cochlear implant 100. Such embodiments may permit the recipient to set their own threshold and comfort levels as well as the MAP for the cochlear implant 100. This may save clinical time by alleviating clinicians of the burden of searching for the optimal or near-optimal MAP for the recipient.

In performing a genetic algorithm search, an audible signal may be provided to a recipient using each of a population of MAPs. The recipient may then identify which MAPs from the population provide the best results. These MAPs identified as providing the best results may then be used to create new population of MAPs (referred to as children of identified parent MAPs). These children MAPs are then used to provide stimulation to the recipient and the recipient then identifies which MAPs provided the best results. Each successive population of MAPs is referred to as a generation. The process of determining the best MAPs and generating children MAPs from these MAPs then continues until some criteria are met, such as a maximum number of iterations has been reached or the difference between the MAPs of a particular generation is below a threshold. In using a MAP to provide stimulation, the dynamic range (e.g., threshold and comfort levels) of each electrode is typically included in the MAP provided by the fitting system 206 to the cochlear implant 100.

Because loudness levels are highly dependent on the aggregate stimulation rate (Rate×Max) of a MAP, as well as the spatial distribution of stimuli, the dynamic range parameters (e.g., threshold and comfort levels) for each MAP may be different. The Rate parameter represents the rate of stimulation, often referred to in units of pulses per second. And, the Max parameter represents the number of applied maxima with the MAP. The applied maxima are also sometimes associated with the channels of stimulation (or stimulation channels).

In an embodiment employing a genetic algorithm, the MAP search may use MAPs having six different stimulation rates, and therefore use six different sets of T and C levels, ignoring the Max parameter. If, however, the Max parameter is taken into account, the number (e.g., 6 different rates) of potential Rate×Max combinations becomes larger. For example, if 6 different rates are possible and each rate may be used with 3 different Max parameters, then the total number of sets of threshold and comfort levels grows to 18 (6×3). If an audiologist is required to determine the thresholds and comfort levels for each of these Rate×Max combinations, this may take a significant amount of time by the audiologist and thus increase the costs of the fitting process. In order to reduce the amount of time required for the genetic algorithm search and to increase effectiveness, a recipient may use the fitting system 206 to measure the threshold and comfort levels associated with the different Rate-Max combinations.

It should be noted that a genetic algorithm search is but one example of a situation where it may be desirable to measure the T and C levels for a variety of Rate×Max combinations, and embodiments may be used in other situations. For example, if a recipient desires to improve the battery performance of their cochlear implant, a clinician may want to test a variety of different Rate×Max combinations to determine a stimulation rate that provides both acceptable battery life and acceptable hearing performance for the user. Or, for example, in an embodiment, the fitting system may measure stimulation level parameter(s) (e.g., T and C levels) for a specified fixed rate and a variable number of maxima, or a variety of rates with a fixed number of maxima, or a fixed rate and number of maxima, or combinations thereof. Further, it should also be noted that measuring stimulation level parameters for different Rate×Max combinations is merely one example for measuring stimulation level parameters. And, in other embodiments, stimulation level parameters (e.g., T and C levels) may be measured for different combinations of other parameters in addition to (or in lieu of) different Rate×Max combinations. For example, stimulation level parameters may be measured for different combinations of different or additional parameters to Rate and Max, such as for example different pulse widths (durations), different pulse shapes, different configurations for implementing multi-electrode channels, different weights assigned to different electrodes in implementing a multi-electrode channel, different stimulation methods (e.g., bipolar, monopolar, multiple monoplolar), etc. The particular combination of parameters for which stimulation level parameters (e.g., T and C levels) are to be measured, may vary, for example, based upon the type of sound processing strategy (e.g., ACE, PACE (aka MP3000)) implemented. For example, measuring stimulation level parameters (e.g., T and C levels) for different Rate×Max combinations may be beneficial in embodiments implementing the ACE sound processing strategy. But, in embodiments, implementing a different sound processing strategy, the fitting system may measure the stimulation level parameters (e.g., T and C levels) for different combinations of different parameters.

The following provides an exemplary method that a fitting system may use in obtaining the threshold and comfort levels for different Rate×Max combinations. As an initial matter, an audiologist or clinician may provide the fitting system 206 with an instruction set for obtaining the threshold and comfort levels for each Rate×Max combination. This instruction set may be provided in the form of an ASCII text file, or, for example, the clinician may provide these instructions to the fitting system 206 using a graphical user interface (not show) adapted for use by the clinician. These instructions may, for example, specify that the fitting system 206 is to measure threshold and comfort levels for every MAP that may potentially be encountered in a MAP search. This MAP search may be, for example, performed using a genetic algorithm. This instruction set as well as an exemplary clinical user interface for providing these instructions will be discussed in further detail below.

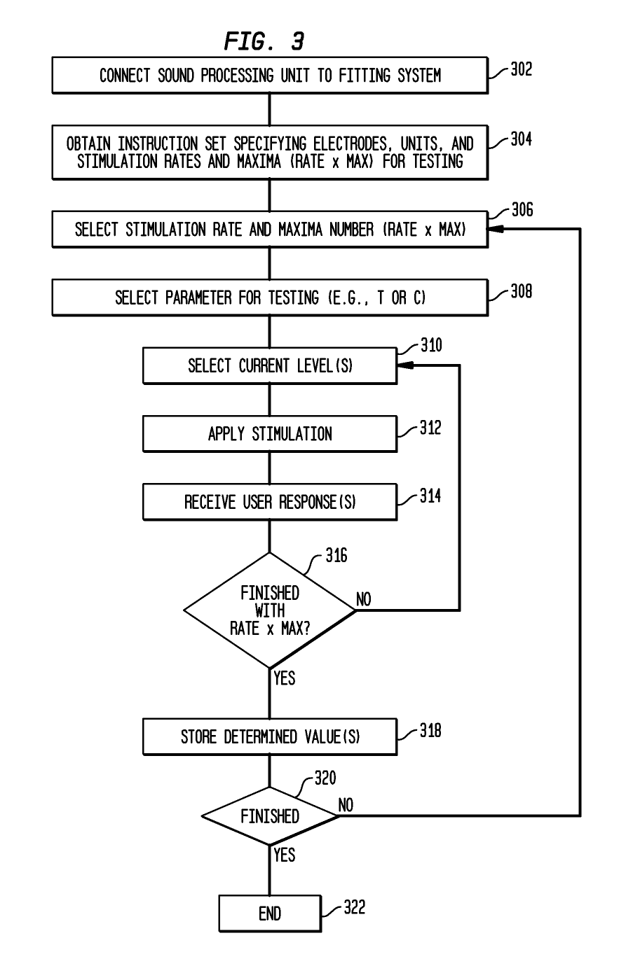

FIG. 3 is a high-level flow chart illustrating operations that may be performed for determining threshold and maximum comfort levels, in accordance with an embodiment. FIG. 3 will be discussed with reference to the fitting system illustrated in FIG. 2.

A recipient 202 may initiate the process by connecting cochlear implant 100 to fitting system 206 at block 302. This may be accomplished by plugging a cable into the speech processor 126 of the cochlear implant 100 and the fitting system 206. Or, for example, fitting system 206 and cochlear implant 100 may connect wirelessly in response to, for example, the recipient entering an instruction via user interface 214 that instructs fitting system 206 to wirelessly initiate a connection with cochlear implant 100. This connection may also cause the fitting system to begin some initialization procedures. These initialization procedures may include a calibration step to help ensure that constant sound level pressure is delivered to the sound processing unit 126 of the cochlear implant 100 by the fitting system 206.

Fitting system controller 212 may then obtain the instruction set for performing the desired measurements, at block 302. As noted above, this instruction set may specify that the fitting system 206 is to obtain a plurality of threshold and maximum comfort level parameters. This instruction set may be stored in a memory or other storage device of fitting system controller 212.

The fitting system controller 212 may then select a Rate×Max combination from amongst the combinations to be tested, at block 304. This initial Rate×Max combination may be specified in the instruction set, the fitting system controller 212 may randomly select one of the combinations, or any other mechanism may be used for selecting this initial combination.

The fitting system controller 212 may also select the parameter to be measured (e.g., T or C levels) at block 308. This parameter may be specified in the instruction set, or determined by the fitting system controller 212 in some other manner. The fitting system controller 212 may then select one or more current level(s) for application of stimulation at block 310. Next, the fitting system controller 212 may direct the cochlear implant 100 to apply stimulation using the specified parameters and Rate×Max combination at block 312. The fitting system controller 212 may then obtain a recipient response at block 314 regarding the recipient's perception of the applied stimulation. This response may be provided by the recipient using input device 224. The fitting system controller 212 may then analyze the obtained response to determine at block 316 if additional testing is to be performed or not. If so, the fitting system controller 212 may store the received response at block 318 and return to block 310 for further testing.

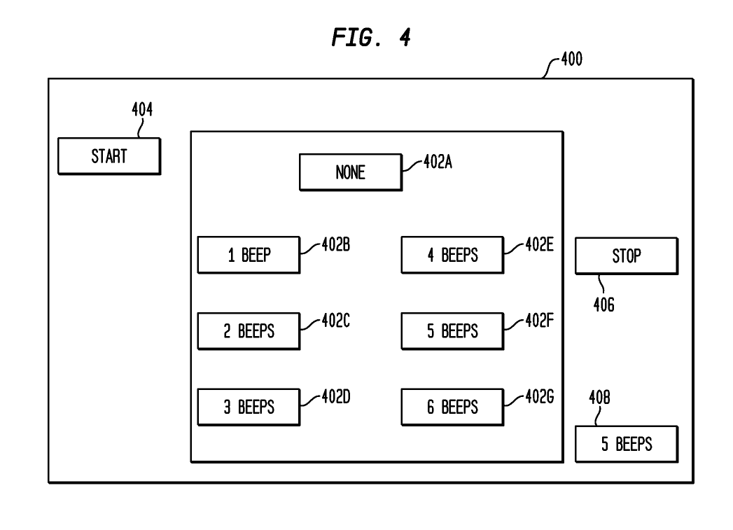

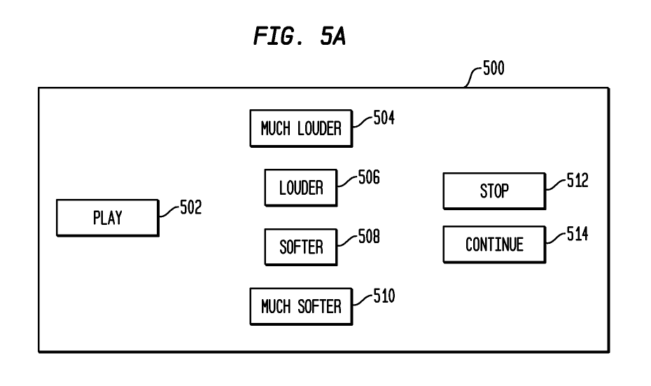

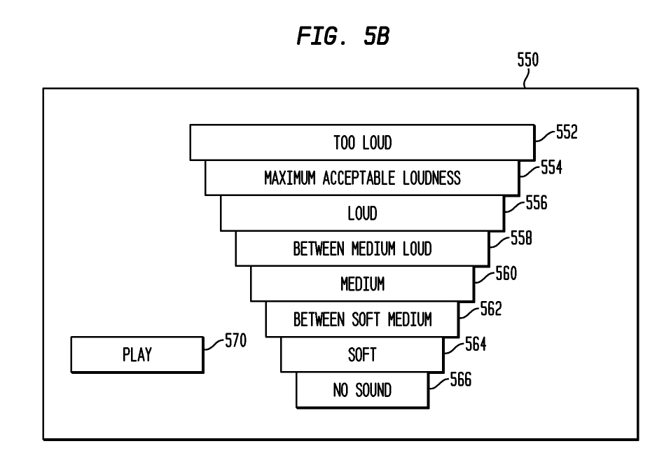

There are various mechanisms that may be employed for selecting current levels (block 308), applying stimulation (block 310), and obtaining a recipient response (block 312). For example, in one embodiment, fitting system controller 212 may randomly select a number (e.g., a random number between 1 and 6) of stimulations to be applied in measuring threshold levels. Fitting system controller 212 may then select the current level for each of the stimulations. The fitting system controller 212 may then transmit information to the cochlear implant 100 to cause the cochlear implant to apply stimulation at each of the determined current levels. Each of these stimulations may be separated in time, such that if the recipient hears each of these stimulations, the recipient would hear a successive group of beeps each with a different loudness. In such an example, the recipient may be provided at block 314 with a GUI for entering their perception of the applied stimulation (e.g., how many beeps they heard). Exemplary GUIs and methods for obtaining parameter measurements using these exemplary GUIs are provided in more detail below with reference to FIGS. 4 and 5.

The fitting system controller 212 at block 320 may then determine if additional testing is to be performed. If so, the process returns to block 306. And, if not, the process ends at block 322. As noted above, the different tests to be performed may be stored in an instruction set or by fitting system controller 212. For example, during the first pass through the process, the fitting system controller 212 may measure the thresholds for one Rate×Max combination. Then, during a second pass through blocks 306 through 320, the comfort level for this Rate×Max combination may be measured. After which, the threshold and comfort levels for a different Rate×Max combination may be measured.

FIG. 4 illustrates an exemplary GUI 400 that may be provided to a recipient for obtaining the recipients perception of applied stimulation, in accordance with an embodiment. As illustrated, GUI 400 may comprise a set of icons 402 that the recipient may select to indicate how many beeps the recipient heard. For example, these icons 402 may include an icon for selecting that the recipient heard zero beeps 402A, one beep 402B, two beeps 402C, three beeps 402D, four beeps 402E, five beeps 402F, and six beeps 402G. This GUI 400 may be displayed on display 222. The recipient may, using input interface 224, select the icon corresponding to the number of beeps heard by the recipient. The input interface 224 may then provide this response to fitting system controller 212.

Additionally, GUI 400 may include a start icon 404 that the recipient may select to direct the fitting system controller 212 to start the application of stimulation. Additionally, the GUI 400 may comprise a stop button 406 that the recipient may select to stop the process, such as if the recipient needs to leave for any purpose. After the user enters their response GUI 400 may also display the correct number of beeps 408.

In response to an incorrect answer, the fitting system controller 212 may increase the current levels by one large step and apply the same number of stimulation signals at the increased current levels. In this manner, the current level quickly ascends to a general audible level (stimulation grows louder by large steps until sound is heard). In response to a correct answer, the fitting system controller 212 may drop the stimulation level to that which was previously inaudible and begin a counted-Ts procedure. The displayed GUI may remain the same throughout this process, and the question “How many beeps did you hear” continues. The counted-T's procedure refers herein to a procedure where the fitting system controller 212 randomly selects at block 310 a number of uniformly distributed beeps (e.g., between 2 and 6), rather than a fixed number. The recipient again chooses between the same buttons labeled “None”, “1”, “2”, “3”, “4”, “5” and “6” 402A-G. In this way, thresholds may be measured more resolutely. With every correct response, the current level is decreased by 2 small steps, and with every incorrect response, the current level is increased by 1 small step at block 410. The task continues until the number of correct responses at any one level meets a particular value (named “Reversals”) at block 416. This reversal value may be specified in the instruction set. Fitting system controller 212 may store the measured final threshold value at block 416. This threshold may be stored in a storage within fitting system controller 212.

After the thresholds for all electrodes to be measured of the cochlear implant 100 are determined, fitting system may check the determined values to see if there are any outliers. For example, threshold values typically follow a rather uniform curve across electrodes. If a measured threshold for a particular Rate×Max combination is found to be significantly above or below this curve, the fitting system 212 may then at block 316 re-measure the threshold for this or an adjacent electrode. This check may be performed, for example, by obtaining the median of all the computed thresholds and then determining if all the thresholds are within +/− a value (n) of the computed median.

Because the threshold values typically follow a rather uniform curve, in embodiments, the fitting system may measure thresholds for only a subset of the electrodes of the electrode array (e.g., 5 out of the 22 electrodes of the electrode array). The fitting system may then use these measured electrodes to interpolate the thresholds for the other electrodes. A further explanation of how thresholds may be interpolated by measuring the thresholds of a subset of the electrode array's electrodes is provided in U.S. patent application Ser. No. 10/518,812 entitled “Parametric Fitting of a Cochlear Implant,” by Guido F. Smoorenburg and filed on Oct. 11, 2005, the entire contents of which are incorporated by reference herein. Or, for example, in an embodiment, the fitting system may use a “streamlined” fitting procedure in which linear interpolation is used (e.g., blind linear interpolation), rather than a curve-fitting technique based on heuristic (not blind) curves. One exemplary “streamlined” technique employing blind linear interpolation is provided in Plant et al., “Evaluation of Streamlined Programming Procedures for the Nucleus Cochlear Implant with the Contour Electrode Array,” Ear and Hearing. 26(6):651-668, December 2005.

As noted above, fitting system 206 may also be used to compute the comfort levels for each Rate×Max combination. FIG. 5A illustrates an exemplary GUI 500 that may be provided to a recipient for measuring the comfort levels, in accordance with an embodiment. As illustrated GUI 500 may include a play icon 502, a much louder icon 504, an louder icon 506, a softer icon 508, a much softer icon 510, a stop button 512, and a continue button 514. The play button 502 instructs the fitting system controller 212 to direct the cochlear implant 100 to apply stimulation using the currently specified T and C value, and the specified Rate×Max combination as well as the other parameters specified in block 304. The louder button 506 increases the current level at block 310 by, for example, one step, and then the fitting system controller 212 at block 312 directs the cochlear implant 100 to apply stimulation at this new current level. The much louder button 504 functions in the same manner as louder button 506, but instead of increasing the current level by one step increases the current level by a larger increment (e.g., 2, 3, 4, etc. steps).

The softer button 508 decreases the current level at block 310 by for example, one step, and then the fitting system controller 212 at block 312 directs the cochlear implant 100 to apply stimulation at this new current level. The much softer button 510 functions in the same manner as softer button 508, but instead of decreasing the current level by one step increases the current level by a larger number of steps (e.g., 2, 3, 4, etc.). The step size as well as the number of step sizes each button may increase or decrease the current level may be specified in the instruction set obtained at block 304. Further, in another example, the large step size may be individually specified and need not be a multiple of the small step size. Further, the step sizes for increasing and decreasing the current level may be the same or different.

The stop button 512 may cause the process to stop, such as, for example, if the recipient needs to leave or otherwise terminate the procedure. The continue button 514 may be used by the recipient to indicate that the maximum comfort level has been reached, and the process should continue to the next measurement at block 316.

This stimulation applied in accordance with GUI 500 may be representative of, for example, a beep at a particular frequency, a music clip, a person or people speaking, etc. Further, GUI 500 may be used to apply stimulation one electrode at a time to set the comfort levels one at a time. Or, for example, a stimulation signal in accordance with live audio may be applied and the current levels of all electrodes adjusted in response to the recipient's selection of one of the icons. This mechanism of using simulated live audio and adjusting the current levels of multiple electrodes simultaneously in response to the recipient's selection may use principals such as those discussed in U.S. patent application Ser. No. 10/518,812 entitled “Parametric Fitting of a Cochlear Implant,” filed on Oct. 11, 2005, which is hereby incorporated herein in its entirety. For example, an initial current level profile may be determined based on the measured threshold levels. These measured threshold levels may be used to fit a curve. Then this curve may be adjusted up or down, or tilted in response to the recipient's selections to obtain the comfort levels.

It should be noted that GUI 500 is but one example of a user interface that may be presented to a recipient for measuring the maximum comfort levels for the recipient. FIG. 5B illustrates another exemplary GUI 550 comprising a different set of icons may be used for measuring the maximum comfort levels. As illustrated, GUI 550 displays a plurality of icons such as icons: Too Loud 552, Maximum Acceptable Loudness 554, Loud 556, Between Medium and Loud 558, Medium 560, Between Soft Medium 562, Soft 564, and No Sound 566. As with GUI 500, the stimulation applied may be representative of, for example, a beep at a particular frequency, a music clip, a person or people speaking, live sound arriving through the microphone of the cochlear implant, etc.

The stimulation may be applied at a particular current level and the recipient may select one of the icons 552-566. If the recipient selects any of the icons other than the Too Loud 552 or Maximum Acceptable Loudness 554 icon, the current level of the applied stimulation may be increased. This current level may be increased by a specified step size such as discussed above with reference to FIG. 5A. Further, this step size may be different depending on which icon is selected by the recipient. For example, if the recipient selects an icon indicating that they perceived no sound 566, the current level may be increased by a larger step size that if the recipient selected the icon labeled Loud 556.

As one example, the step sizes for the icons may be set such that the Loud icon 556 increases the current level by 1 step, the Between Medium and Loud icon 558 increases the current level by 2 steps, the Medium icon 560 increases the current level by 3 steps, the Between Soft Medium icon 562 increases the current level by 4 steps, the Soft icon 564 increases the current level by 5 steps, and the No Sound icon 566 increases the current level by 6 steps.

If the recipient selects the Too Loud icon 552, the current level may be reduced by a specified step size (e.g., 2 steps). And, if the recipient select the Maximum Acceptable Loudness icon 554, the fitting system may determine that the maximum comfort level for the recipient for this electrode and Rate×Max combination is equal to the current level of the applied stimulation.

GUI 550 also includes an icon labeled play 570. The user may select the play icon 570 to begin the process of measuring the maximum comfort levels for the recipient. After the user selects the play icon 570, the icon may replaced with an icon labeled stop that a recipient may use to terminate the process, such as discussed above with reference to the stop icon 512 of GUI 500.

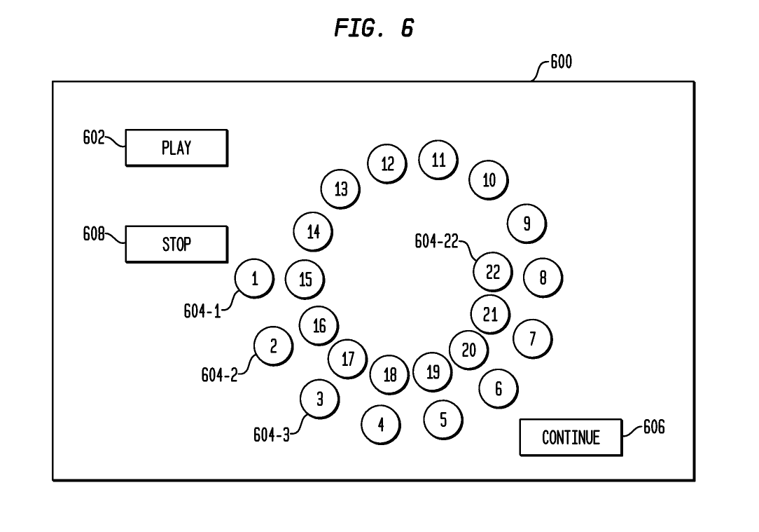

Fitting system 206 may also be used to identify individual electrodes that are either too loud or too soft, compared to other electrodes. FIG. 6 provides an exemplary GUI 600 that may be used to individually adjust electrode current levels, in accordance with an embodiment. Fitting system 206 may provide this GUI 600 to the recipient after determining the threshold and comfort levels. For ease of explanation, GUI 600 will be referred to as a Sweep GUI 600. As illustrated, Sweep GUI 600 may provide an icon 604-1 through 604-22 corresponding to each electrode of the electrode array of cochlear implant 100. Sweep GUI 600 may further comprise a play button 602, a continue button 606, and a stop button 608.

The play button 602 may be used to direct fitting system 206 to sweep through (e.g., sequentially) all or a subset of all electrodes at a particular current level, such as for example, the measured comfort level for the electrode, or a particular number of step sizes below the comfort level. As each electrode is played (i.e., stimulation applied via the electrode) the electrode being stimulated may be highlighted on the GUI 600. This may be accomplished, for example, by changing the color of the icon, changing its size or shape, a combination thereof, or any other mechanism. Electrodes identified by the recipient as too loud or too soft, in relation to their neighbors, may be selected by the recipient clicking-on the icon 604 corresponding to the electrode. The fitting system 206 may then change the color of the selected icon to a particular color (e.g., red), or its size, shape, a combination thereof or another mechanism may be used to highlight the selection of the electrode. The recipient may select the continue button 606 to advance to a balancing GUI that may be used to balance the current level of the selected electrode. The stop button 608 may be used to stop the process.

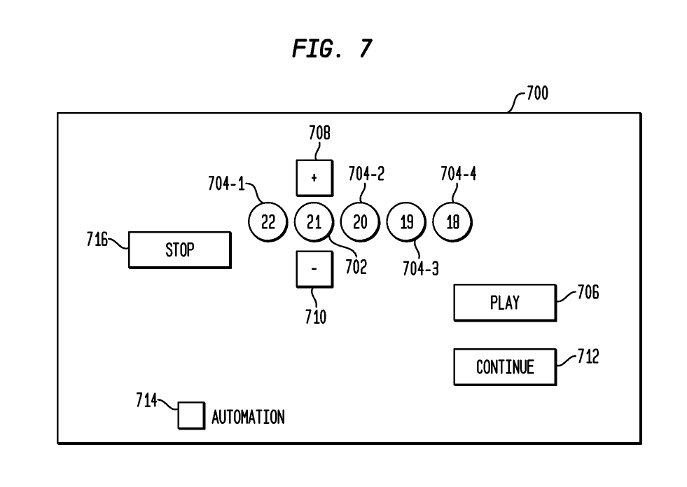

FIG. 7 illustrates an exemplary GUI 700 that may be used for balancing an electrode, in accordance with an embodiment. Balancing GUI 700 may be used, for example, by a recipient to set all electrodes to the same loudness level. GUI 700 displays an icon 702 representative of the electrode identified by the recipient using sweep GUI 600 as either too loud or too soft. The selected electrode icon 702 is situated among a number, for example four, other electrode icons 704-1 through 704-4 representative of electrodes that were not identified. These other electrodes may be selected so that some, for example two, are below the selected electrode and some, for example two, are above the selected electrode. If, however, there is only one electrode above or below the selected electrode, then only that electrode may appear above or below the selected electrode. Or, if there are no electrodes above or below, then only electrodes from the side in which there are electrodes may be used.

In the exemplary GUI 700, only one icon 704-1 is illustrated to the left of icon 702 and three icons 704-2 thru 4 are illustrated to the right of icon 702. GUI 700 also illustrates a Play button 706 that causes fitting system 206 to sequentially apply stimulation on each of the electrodes corresponding to the displayed icons 702 and 704. The Louder button 708 may be selected by the recipient to increase the level (T, C, or both) of the selected electrode by a small level increment (e.g., one step), process the audio with the new levels, and then present the five electrodes sequentially. The Softer button 710 may be selected by the recipient to decrease the level (T, C, or both) of the selected electrode by a small level increment (e.g., one step), process the audio with the new levels, and then present the five electrodes sequentially. The Continue button 712 may be selected by the recipient to progress to a screen for balancing the next selected electrode, if one exists. Otherwise, it initiates the next command in the automation protocol if, for example, the “Automation checkbox” 714 is checked. If automation is not currently selected (the “Automation checkbox” 714 is not checked), the task/graphics screen is emptied. GUI 700 may also comprise a Stop button 716 to terminate the procedure.

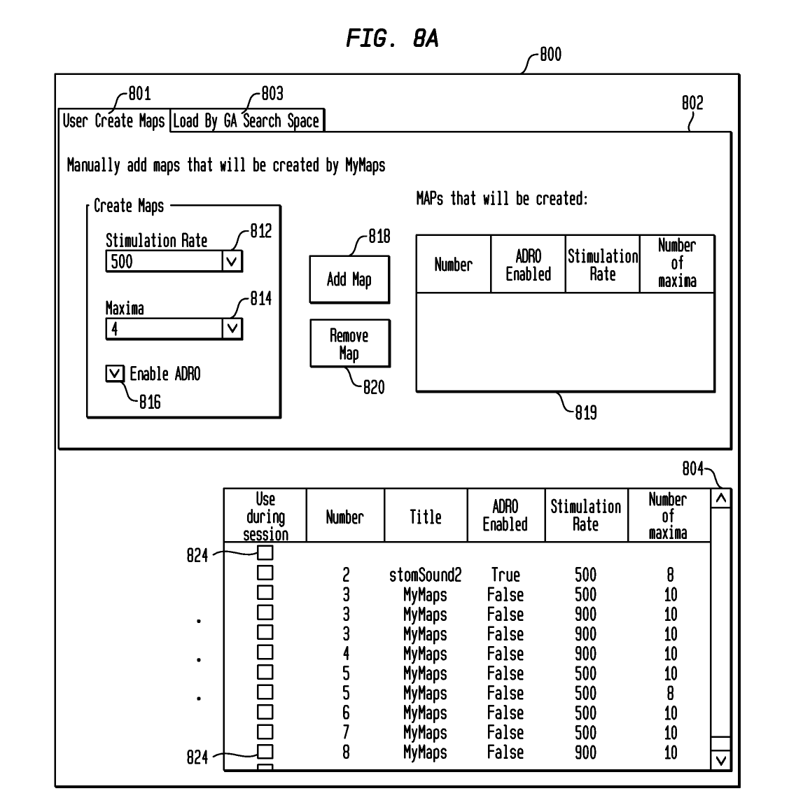

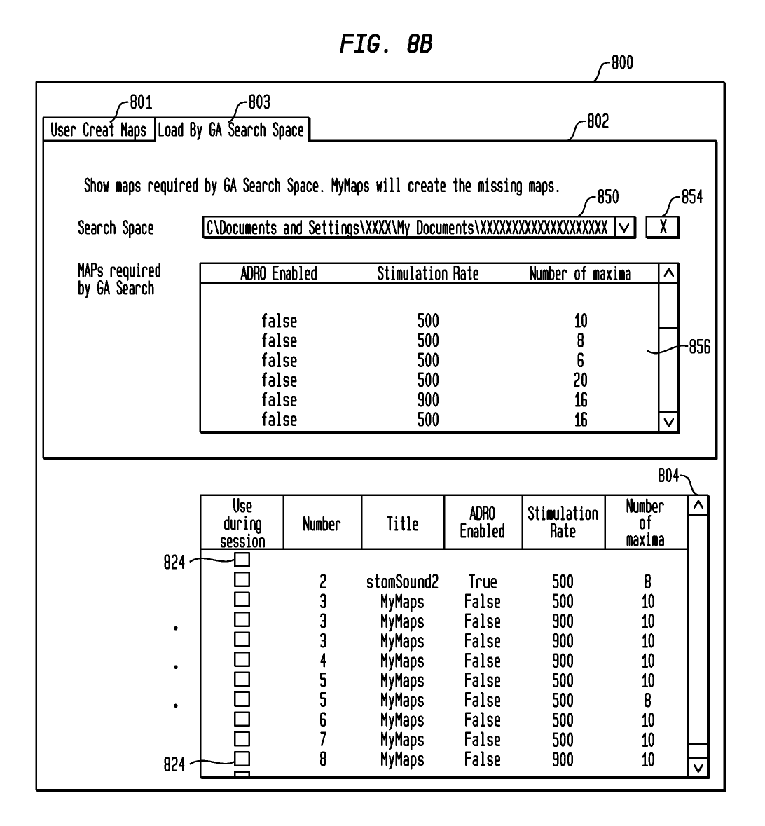

As noted above, in embodiments, an instruction set may be used by the fitting system 206 to specify the type of measurements to be conducted as well as the parameters for these measurements. This instruction set may be included in a file, such as an ASCII file created by an audiologist or clinician and stored by fitting system 206. This file may be created using, for example, a clinical GUI or, for example, created using a simple text editor and then stored in fitting system controller 212. Exemplary clinical GUIs will be discussed in more detail below with reference to FIGS. 8 and 9.

This instruction set file may use various commands to instruct the fitting system controller 212 to perform different steps. The following provides a description of exemplary commands that may be used in the instruction set file. In this example, commands are specified in the instruction set file by using the word “Next” followed by the command. That is the term “Next” acts as a flag and indicates that a command follows. Any line in the instruction set file that does not contain the “Next” precursor will be ignored in this example. The exemplary commands may include the following: Create, Find, Derive, Psych, Live, Sweep, Units, Adjust, Save, Parity, and Implant. A description of each of these exemplary commands follows.

The “Create” command causes the creation of a new set of Ts and Cs, or if the MAP already exists, rather than creating a new MAP with T and C levels initialized to zero, the existing MAP will be loaded. This MAP may then become the Current MAP. The syntax may be as follows: Next Create Strat Rate Max, where Strat specifies the processing strategy (e.g, ACE or PACE), Rate specifies the channel-specific stimulus rate, Max specifies the number of selected maxima. This command may be used, for example, as follows: Next Create ACE 2400 6, Next Create PACE 900 4.

The “Find” command instructs the fitting system to search through the existing MAPs in search of the designated MAP, which becomes the Current MAP. The syntax may be as follows: Next Find Strat Rate Max, where Strat specifies the processing strategy (e.g, ACE or PACE), Rate specifies the channel-specific stimulus rate, Max specifies the number of selected maxima. This command may be used, for example, as follows: Next Find ACE 2400 6, or Next Find PACE 900 4.

The “Derive” command instructs the fitting system to create a new set of Ts and Cs, based on an existing MAP. This may be useful for adjusting levels in the case of increasing the number of maxima or changing the strategy, yet keeping the same stimulation rate. In response to the “Derive” command, the fitting system looks among the existing MAPs for specified existing MAP, and derives a new MAP from this existing MAP. If it does not exist, a new MAP will be created, but the T and C levels will be initialized to zero. This derived MAP becomes the Current MAP. In an embodiment, the derived MAPs will have the same T levels as the original MAPs, but C levels will be 5 current levels below the C levels of the original MAP. The syntax may be as follows: Next Derive origStrat origRate origMax newStrat newMax, origStrat specifies the strategy (e.g., ACE or PACE) of the MAP to find, origRate specifies the channel-specific stimulus rate of the original MAP, origMax specifies the number of maxima of the original MAP, newStrat specifies the strategy (e.g., ACE or PACE) of the new MAP, and newMax specifies the number of maxima of the new MAP. For safety considerations, in an embodiment, ACE MAPs may not be derived from PACE MAPs. This command may be used, for example, as follows: Next Derive ACE 1200 8 PACE 8, or Next Derive PACE 1200 8 PACE 4

The “Psych” command may instruct the fitting system to initiate psychophysical measurement of levels pertaining to the current MAP. The syntax may be as follows: Next Psych Level, where Level specifies the type of level (e.g., T or C) to be presented and adjusted. This command may be used, for example, as follows: Next Psych Ts, or Next Psych Cs.

The “Live” command may instruct the fitting system to initiate live-voice measurement of levels pertaining to the current MAP. In an embodiment, the audio used may be an attempt to re-create live-voice by using 4-talker babble. The syntax may be as follows: Next Live adjLevel, where adjLevel specifies the type of level (e.g., T or C) to be presented and adjusted. This command may be used, for example, as follows: Next Live Ts, or Next Live Cs.

The “Sweep” command instructs the fitting system to initiate the Sweep and Balance procedure on the current MAP, such as was discussed above with respect to FIGS. 6 and 7. As discussed above, during sweep, the fitting system individually presents the electrodes (i.e., applies stimulation on the electrode) and the recipient may individually select electrodes that are either too loud or too soft in relation to the other electrodes in the array. During balancing, the fitting system may adjust selected electrodes similarly to how adjustments are accomplished in conventional psychophysical measurement. The syntax may be as follows: Next Sweep presLevel adjLevel, where presLevel specifies the presentation level as a percentage of the dynamic range, and adjLevel specifies the levels being adjusted. This command may be used, for example, as follows: Next Sweep 100 Cs, or Next Sweep 50 Ts.

The “Units” command changes the type of units used during an adjustment (either current levels or % dynamic range) and changes the step sizes of the small and large steps. These steps may also be referred to herein as small and large increments. The syntax may be as follows: Next Units Type Small Large #Beeps Duration Reversals, where Type specifies the type of units (e.g., either current level (CL) or dynamic range (DR)), Small specifies the small increment size (e.g., from 1 to 5), Large specifies the large increment size (e.g., from 5 to 50), #Beeps specifies the number of psychophysical stimulus repetitions (e.g., from 1 to 6), Duration specifies the duration of stimulus (from 0.01 to 5.00), Reversals specifies the number of psychophysical task's negative reversals (e.g., from 0 to 5). This command may be used, for example, as follows: Next Units CL 2 10 6 .5 0, or Next Units DR 5 50 1 5 3

The “Adjust” command may instruct the fitting system to shift the level (e.g., T or C) on any or all electrode. This command may have the following syntax: Next Adjust Type Electrode Inc/Dec, where Type specifies which level will be modified (e.g., T, C or both T and C (TC)), Electrode specifies which electrode will be modified (e.g., Elec# or All), Inc/Dec identifies if the level is incremented or decremented. This command may be used, for example, as follows: Next Adjust T 3 2, or Next Adjust 50 TC All-4.

The “Save” command may be used to instruct the fitting system to save the current MAP into a database. The syntax may be as follows: Next Save.

The “Parity” command may instruct the fitting system to set the C-level profile to match the T-level profile. The term profile refers to the collection of determined levels (e.g., T or C levels) for the recipient's electrode array. The syntax may be as follows: Next Parity.

The “Implant” command may specify the recipient's specific implant type. The syntax may be as follows: Next Implant Type, where Type specifies the type of implant. This command may be used as follows: Next Implant cic3, or Next Implant cic4.

The following provides an exemplary instruction set using the above-discussed commands that may be used to define the operations for measuring the levels, such as was discussed above with reference to FIG. 3.

Next Implant cic4
Next Electrode 1 5
Next Units CL 3 7 1 .4 3
----- Base MAPs ------------------------------------
Next Create ACE 900 16
Next Units CL 3 7 3 .4 3
Next Psych Ts
Next Parity
----- 900 PPS ------------------------------------
Next Find ACE 900 16
Next Units CL 3 7 3 .4 1
Next Adjust Cs All INC
Next Psych Cs
Next Units DR 3 40 1 3 1
Next Adjust Cs All DEC
Next Units CL 3 7 1 3 1
Next Live Cs
Next Units CL 3 7 1 .4 1
Next Sweep 100 Cs
Next Sweep 50 Ts
Next Clear
Next Save

As noted above, in embodiments, fitting system 206 may also comprise a clinical user interface for use by an audiologist or clinician. This clinical interface may be used, for example, to specify the parameters for testing, such as the parameters included in the above-discussed instruction set. Additionally, in embodiments the clinical user interface may be also be used by the audiologist or clinician to adjust the measured levels after completion of the measurement process by the recipient. Additionally, the clinical user interface may enable the audiologist/clinician to create MAPs (e.g, ACE or PACE MAPs) having different stimulation rates and numbers of maxima. That is, these created MAPs may have different Rate×Max combinations. The fitting system 206 may then determine the levels (e.g., T and C levels) for these MAPs (ie., Rate×Max). The clinical user interface may also permit the audiologist/clinician to set various testing parameters for the measurements, such as, for example, the electrodes to use during the measurements, the number of reversals used by the fitting system in obtaining the levels using psychophysical measurements, the increment and decrement step sizes, the duration of the applied stimulation, etc.

FIGS. 8A and 8B illustrates an exemplary clinical graphical user interface 800 that may used to add a MAP for level (e.g., T and C) measurement, in accordance with an embodiment. As illustrated, interface 800 may comprise a portion 802 for adding a MAP, and a portion 840 for listing MAPs already created and stored by fitting system 206, such as, for example, in a MAP database. The portion for adding MAPs 802 may comprise a tab 801 for creating new MAPs, and a tab 803 for searching for existing MAPs. FIG. 8A illustrates clinical interface 800 when tab 801 is selected. As illustrated, when tab 801 is selected, portion 802 may comprise pull-down 812 for specifying the stimulation for the new MAP, a pull-down 814 for specifying the number of maxima for the new MAP, and a checkbox 816 for selecting whether Adaptive Dynamic Range Optimization (ADRO) should be enabled or not for the new MAP. Additionally, portion 802 may comprise an add MAP button 818 for directing the fitting system to add the MAP with the specified stimulation rate and number of maxima to the MAP database. When the clinician or audiologist presses the add MAP button 818, the MAP is first added to block 819 which lists the MAPs that the fitting system is to create. Portion 802 may also include a remove MAP button 820 for deleting MAPs from block 819.

Portion 804 may list each of the previously created MAPs, as well as a check box 824 corresponding to each listed MAP. The clinician or audiologist may check the corresponding checkbox 824 for each MAP for which the fitting system 206 is to obtain the levels. Interface 800 may also comprise a Go button 830 that the clinician or audiologist may select to instruct the fitting system to obtain the levels for the MAPs created in block 819 as well as those in portion 804 in which the corresponding checkbox 824 is checked. In response to selection of Go button 830, fitting system controller may create and store an instruction set that may be used for measuring the stimulation level parameters for the specified Maps.

FIG. 8B illustrates clinical interface 800 when tab 803 is selected. As illustrated, when tab 803 is selected, portion 802 includes a box 850 permitting the clinician or audiologist to enter, for example, a location for a file including the MAPs that are to be searched. Portion 802 may also include a browse button 854 that the clinician may press to locate such a file. This browse button 854 may function in a similar manner to browse buttons commonly used in computer based systems. These MAPs may then be displayed in block 856. When Go button 830 is selected, fitting system 206 may create an instruction set for obtaining the levels from the MAPs identified in block 856 as well as any in portion 804 in which the corresponding checkbox 824 is checked. This instruction set may be used by the fitting system for obtaining the levels, such as was discussed above with reference to FIG. 3. Or, for example, the information specifying the MAPs may be saved and used, for example, to create an instruction set after other parameters are specified.

It should be noted that GUIs 800 is exemplary only and provided to illustrate one example of a clinical interface that may be used to specify the parameters for the measurements to be performed in obtaining the stimulation level parameters (e.g., T or C levels). And, other user interfaces may be used. For example, the buttons, pull-downs, etc. may be organized in a different manner, or different buttons, etc. may be used, without departing from the invention. Further, the clinical user interface may use the same display 214 and input interface 224 used by recipient 202, or, for example, a separate display and input interface may be used.

It should be noted that although the above-discussed embodiments were discussed with reference to a cochlear implant, in other embodiments a fitting system may be used to permit a recipient to measure the stimulation level parameters of other stimulating medical devices, such as, for example, bone conduction devices, auditory brain stimulators, etc.

Various implementations of the subject matter described, such as the embodiment of FIG. 2, components of may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device

These computer programs (also known as programs, software, software applications, applications, components, or code) include machine instructions for a programmable processor, and may be implemented in a high-level procedural and/or object-oriented programming language, and/or in assembly/machine language. As used herein, the term “machine-readable medium” refers to any computer program product, computer-readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. Similarly, systems are also described herein that may include a processor and a memory coupled to the processor. The memory may include one or more programs that cause the processor to perform one or more of the operations described herein.

All documents, patents, journal articles and other materials cited in the present application are hereby incorporated by reference.

Embodiments of the present invention have been described with reference to several aspects of the present invention. It would be appreciated that embodiments described in the context of one aspect may be used in other aspects without departing from the scope of the present invention.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart there from.

What is claimed is:

1. A method for fitting a stimulating medical device to a recipient, comprising:
transmitting a signal to cause the stimulating medical device to apply stimulation to the recipient;
displaying a graphical user interface to the recipient;
receiving a response to the applied stimulation from the recipient via the graphical user interface;
determining a stimulation level parameter using the recipient's response; and
transmitting the stimulation level parameter to the stimulating medical device for use in applying stimulation to the recipient.

2. The method of claim 1, wherein displaying a graphical user interface comprises:
displaying a graphical user interface to the recipient comprising a plurality of icons, each icon corresponding to a particular perception by the recipient regarding the applied stimulation; and
wherein receiving a response comprises:
receiving an indication that the recipient selected a particular icon displayed by the graphical user interface.

3. The method of claim 1, further comprising:
wherein transmitting a signal comprises:
individually transmitting a plurality of signals, each in accordance with a stimulation rate selected from a set of a plurality of stimulation rates; and
wherein receiving a response comprises:
receiving a response for each of the plurality of signals.

4. The method of claim 3, wherein each of the plurality of stimulation rates are specified by a clinician prior to transmitting the signals.

5. The method of claim 4, wherein each of the plurality of stimulation rates are stored prior to transmitting the signals in a file.

6. The method of claim 5, wherein the file further specifies one or more electrodes of the stimulating medical device for use in applying the stimulation for one or more of the stimulation rates.

7. The method of claim 1, wherein the stimulation level parameter is one or more of a threshold for a stimulation channel, and a maximum comfort level for the stimulation channel.

8. The method of claim 1, further comprising:
executing a genetic algorithm using the determined stimulation level parameter to select values for a subset of one or more parameters selected from a plurality of parameters for which values are to be selected to fit the stimulating medical device, wherein the genetic algorithm is operable to generate one or more successive generations of values for the parameter subset; and
determining based on patient feedback, the values for the parameter subset in each of the one or more successive generations.

9. A system for fitting a stimulating medical device to a recipient, comprising:
a fitting system controller configured to transmit a signal to cause the stimulating medical device to apply stimulation to the recipient;
a display configured to display a graphical user interface to the recipient; and
an input device configured to receive a response from the recipient, using the graphical user interface, regarding stimulation applied by the stimulating medical device;
wherein the fitting system controller is further configured to determine a stimulation level parameter using the received response, and transmit the determined stimulation level parameter to the stimulation medical device for use in applying stimulation.

10. The system of claim 9,
wherein the graphical user interface is configured to display a plurality of icons, each icon corresponding to a particular perception by the recipient regarding the applied stimulation; and
wherein the fitting system controller is further configured to receive an indication that the user selected a particular icon displayed by the user interface.

11. The system of claim 9, wherein the fitting system is further configured to individually transmit a plurality of signals, each in accordance with a stimulation rate selected from a set of a plurality of stimulation rates, and receive a response for each of the plurality of signals.

12. The system of claim 11, wherein the fitting system controller further comprises a storage configured to store information regarding a plurality of stimulation rates to be used in transmitting the signals.

13. The system of claim 12, wherein the file further specifies one or more electrodes of the stimulating medical device for use in applying the stimulation for one or more of the stimulation rates.

14. The system of claim 9, wherein the stimulation level parameter is one or more of a threshold for a stimulation channel, and a maximum comfort level for the stimulation channel.

15. The system of claim 9, wherein the fitting system is further configured to execute a genetic algorithm using the determined stimulation level parameter to select values for a subset of one or more parameters selected from a plurality of parameters for which values are to be selected to fit the stimulating medical device, wherein the genetic algorithm is operable to generate one or more successive generations of values for the parameter subset; and determine based on patient feedback, the values for the parameter subset in each of the one or more successive generations.

16. A system for fitting a stimulating medical device to a recipient, comprising:
means for transmitting a signal to cause the stimulating medical device to apply stimulation to the recipient;
means for displaying a graphical user interface to the recipient;
means for receiving a response to the applied stimulation from the recipient via the graphical user interface;
means for determining a stimulation level parameter using the recipient's response; and
means for transmitting the stimulation level parameter to the stimulating medical device for use in applying stimulation to the recipient.

17. The system of claim 16,
wherein the means for displaying a graphical user interface to the recipient comprises:
means for displaying a graphical user interface comprising a plurality of icons, each icon corresponding to a particular perception by the recipient regarding the applied stimulation; and
wherein the means for receiving a response comprises:
means for receiving an indication that the user selected a particular item displayed by the user interface.

18. The system of claim 16, further comprising:
wherein the means for transmitting a signal comprises:
means for individually transmitting a plurality of signals, each in accordance with a stimulation rate selected from a set of a plurality of stimulation rates; and
wherein the means for receiving a response comprises:
means for receiving a response for each of the plurality of signals.

19. The system of claim 18, further comprising:
means for storing information regarding a plurality of stimulation rates to be used in transmitting the signals.

20. The system of claim 19, wherein each of the plurality of stimulation rates are stored prior to transmitting the signals in a file.

21. The system of claim 20, wherein the file further specifies one or more electrodes of the stimulating medical device for use in applying the stimulation for one or more of the stimulation rates.

22. The system of claim 16, wherein the stimulation level parameter is one or more of a threshold for a stimulation channel, and a maximum comfort level for the stimulation channel.

23. The system of claim 16, further comprising:
means for executing a genetic algorithm using the determined stimulation level parameter to select values for a subset of one or more parameters selected from a plurality of parameters for which values are to be selected to fit the stimulating medical device, wherein the genetic algorithm is operable to generate one or more successive generations of values for the parameter subset; and
means for determining based on patient feedback, the values for the parameter subset in each of the one or more successive generations.

24. A computer readable medium comprising a computer program for controlling a processor to execute a method for fitting a stimulating medical device to a recipient, comprising:
transmitting a signal to cause the stimulating medical device to apply stimulation to the recipient;
displaying a graphical user interface to the recipient;
receiving a response to the applied stimulation from the recipient via the graphical user interface;
determining a stimulation level parameter using the recipient's response; and
transmitting the stimulation level parameter to the stimulating medical device for use in applying stimulation to the recipient.
